(19) United States
(12) Patent Application Publication
(10) Pub. No.: US 2004/0014972 A1
(43) Pub. Date: Jan. 22, 2004

(54) Arylpiperazine derivatives and their use as psychotropic agents

(76) Inventors: Rudolf Gottschlich, Reinheim (DE); Dieter Dorsch, Ober-Ramstadt (DE); Gerd Bartosyk, Weiterstadt (DE); Jurgen Harting, Darmstadt (DE); Christoph Seyfried, Seeheim-Jugenheim (DE); Christoph van Amsterdam, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/363,168

(22) PCT Filed: Aug. 7, 2001
(86) PCT No.: PCT/EP01/09108

(30) Foreign Application Priority Data
Sep. 5, 2000 (DE) 100-43-659.5

(51) Int. Cl. C 07D 4 3/02

(52) U.S. Cl. 544/235; 544/363; 544/284

(57) ABSTRACT

The invention relates to arylpiperazine derivatives of formula (I), wherein R¹, R², A, B, Ar and n have the given meanings.

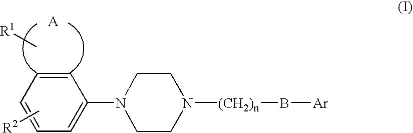

The invention relates to arylpiperazine derivatives, their preparation and their use as psychopharmaceuticals.

The arylpiperazine derivatives according to the invention can be represented by the general formula I

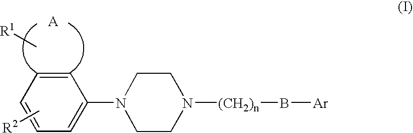

where

A is a fused heteroaromatic or heteroaliphatic ring comprising one or two nitrogen atoms,

B is —CO— or —CHOH— or —C(Ar)(OH)—

R¹ and R² independently of one another are H, alkyl, C₁-C₆ or halogen

Ar is phenyl or thiophene, which is unsubstituted or monosubstituted or polysubstituted by halogen, NO₂ or CN and

n is 1, 2, 3 or 4,

and their salts and solvates.

Psychoses, which also include diseases of the schizophrenia type, have been attributed to a hyperactivity of the limbic dopamine system (Snyder et al., Science 184: 1243-1253, 1974). The antipsychotic effect of neuroleptics has been attributed to their D₂-antagonistic properties (with regard to the nomenclature of the receptors: Basic Neurochemistry, Editors: G. J. Siegel, B. W. Agranoff, R. W. Albers, P. B. Molinoff, 5th edition, Raven Press, Ltd, N.Y. USA, Chapters 12 and 13; otherwise the following technical publications: Creese et al., Science 192: 481-483, 1976; Farde et al., Psychopharmacology 99: 28-31, 1989; Feeman et al., Nature 261: 717-719, 1976; Wiesel et al., Prog. Neuro-Psychopharmacol. & Biol. Psychiat. 14: 759-767, 1990). Consequently, the classical dopamine hypothesis of schizophrenia was formulated, according to which neuroleptics have to bind to the D₂ receptor. On account of their extrapyramidal side effects, the employment of classical D₂ antagonists is severely restricted, especially in the case of chronic administration. The extrapyramidal side effects include, for example, tremor, akinesia, dystonia and akathisia (Cavallaro & Smeraldi, CNS Drugs 4: 278-293, 1995). There are only a few antipsychotics which cause significantly fewer or no extrapyramidal side effects at all and which are described as “atypical neuroleptics” (Kervin, Brit. J. Psychiatry 1964, 141-148, 1994). The prototype atypical neuroleptic clozapine has extremely low extrapyramidal side effects, but causes other serious complications such as agranulocytosis, which sometimes is fatal (Alvir et al., New Engl. J. Med. 329: 162-167, 1993).

Because 5-HT_{1A} agonists intensify antipsychotic properties of conventional dopamine D₂ antagonists in animals (Wadenberg & Ahlenios, J. Neural. Transm. 74: 195-198, 1988) and prevent the catalepsy induced by dopamine D₂ antagonists (Costall et al., Neuropharmacology 14: 859-868, 1975), 5-HT_{1A}-agonistic properties could be advantageous. The efficacy of buspirone, a pharmacon having 5-HT_{1A}-agonistic and dopamine D₂-antagonistic properties, has been demonstrated in schizophrenia patients (Goff et al., J. Clin, Psychopharmacol. 11: 193-197, 1991). Apart from various dopamine autoreceptor agonists which also have a significant affinity for the 5-HT_{1A} receptor (e.g. U-86170F, Lahti et al., Naunyn-Schmiedeberg's Arch. Pharmacol. 344: 509-513, 1991), PD1431188 (Melzer et al., J. Pharmacol. Exp. Ther. 274: 912-920, 1995) and roxindole (Bartoszyk et al., J. Pharmacol., Exp. Ther. 276: 41-48, 1996), only a few dopamine D₂ antagonists have been developed which also have an affinity for the 5-HT_{1A} receptor, such as mazapertine (Reiz et al., J. Mid. Chem. 37: 1060-1062, 1994), S16924 (Millan et al., Br. J. Pharmacol. 114: 156 B, 1995) or ziprasidone (Seeger et al., J. Pharmacol. Exp. Ther. 275: 101-113, 1995). These already known compounds have disadvantages with respect to affinity or specificity. Thus mazapertine also shows an affinity for the α₁ receptor. S16924 additionally has 5-HT_{2A/C}-antagonistic properties and ziprasidone moreover binds to the 5-HT_{1D/2A/2C} receptors.

It is the object of the invention to make available medicaments, in particular psychopharmaceuticals. It is a further object of the invention to make available compounds which bind both to the dopamine D₂ receptor and to the 5-HT_{1A} receptor.

This object is achieved by the compounds of the general formula I and by their tolerable salts and solvates (see above).

It has been found that the compounds of the formula I and their salts have very valuable pharmacological properties together with good tolerability. They especially act on the central nervous system. They have, in particular, a high affinity for receptors of the 5-HT_{1A} type and/or of the dopamine D₂ type.

Compounds of the formula I are particularly preferably simultaneously agonists of the 5-HT_{1A} receptor and antagonists of the D₂ receptor. Binding to additional 5-HT_{1D/2A/2C} receptors is not observed.

Binding properties of the compounds of the formula I can be determined by known 5-HT_{1A} (serotonin) binding test and dopamine binding tests; (5-HT_{1A} (serotonin) binding test: Matzen et al., J. Med. Chem., 43, 1149-1157, (2000) in particular page 1156 with reference to Eur. J. Pharmacol.: 140, 143-155 (1987); dopamine binding tests: Böttcher et al., J. Med. Chem.: 35, 4020-4026, (1992) with reference to J. Neurochem.: 46, 1058-1067 (1986)).

The compound of the formula I differs from the abovementioned atypical neuroleptics.

The compounds according to the invention can be employed for the treatment of diseases which are associated with the serotinin and dopamine neurotransmitter system and in which high-affinity serotinin receptors (5-HT_{1A} receptors) and/or dopamine D₂ receptors are involved. The most important indication for the administration of the compound of the general formula I are psychoses of any type, in particular also mental disorders of the schizophrenia type. Moreover, the compounds can also be employed for the reduction of cognitive functional disorders, i.e. for improvement of the learning ability and of the memory. The compounds of the general formula I are also suitable for the control of the symptoms of Alzheimer's disease. The substances of the general formula I according to the invention are moreover suitable for the prophylaxis and control of cerebral infarcts (cerebral apoplexy), such as cerebral stroke and cerebral ischaemia. The substances are also suitable for the treatment of disorders such as pathological anxiety states, overexcitation, hyperactivity and attention disorders in children and adolescents, deep-seated developmental disorders and disorders of social behaviour with mental retardation, depression, compulsive disorders in the narrower (OCD) and wider sense (OCSD), certain sexual function disorders, sleep disorders and eating disorders, and also such psychiatric symptoms in the context of senile dementia and dementia of the Alzheimer type, i.e. diseases of the central nervous system in the widest sense.

The compounds of the general formula I and their tolerable salts and solvates can thus be employed as active ingredients of medicaments such as anxiolytics, antidepressants, neuroleptics and/or antihypertensives.

Ar is preferably a phenyl group which is optionally mono-, di-, tri-, tetra- or pentasubstituted by one or more groups Hal, —NO₂ or —CN. Ar can furthermore carry the meaning of a thiophenyl group which is optionally mono- or disubstituted by one or more of the groups Hal, NO₂ or —CN. Ar is in particular fluorophenyl, difluorophenyl, cyanophenyl or tolyl. Very particularly preferably, Ar has the meaning 3-fluorophenyl, 2,4-difluorophenyl, 3-cyanophenyl or 4-fluorophenyl, in particular 4-fluorophenyl.

B preferably carries the meaning —CO— or —C(Ar)(OH)—, in particular —C(4-fluorophenyl)(OH)—.

R¹ and R² are, independently of one another, preferably H or C₁-C₆-alkyl, where 1 to 7 hydrogen atoms are optionally replaced by fluorine. R¹ and/or R² can be branched or unbranched and is preferably methyl, ethyl, propyl, isopropyl, n-butyl, sec-butyl, tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbuytyl, 1- or 2-ethylbutyl, 1-ethyl-1-methylpropyl, 1-ethyl-2-methylpropyl, 1,1,2- or 1,2,2-trimethylpropyl. Particularly preferably, R¹ and/or R² is methyl, ethyl, isopropyl, n-propyl, n-butyl or tert-butyl.

Compounds of the formula I are also particularly preferred in which R¹ and R² are simultaneously H, and compounds of the formula I in which R¹ has the meaning alkyl and R² has the meaning H.

The group

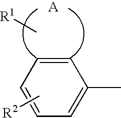

preferably has one of the following meanings:

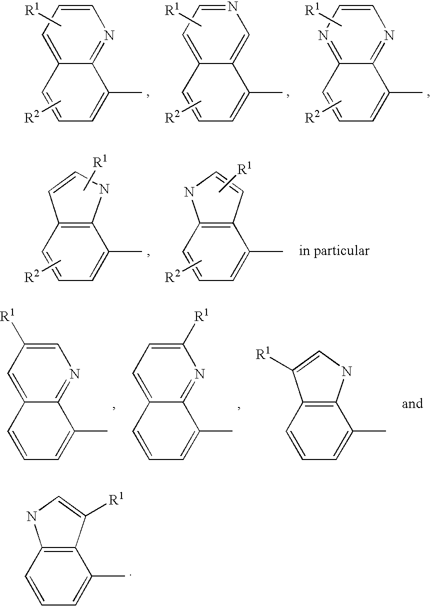

Very particularly preferred meanings are

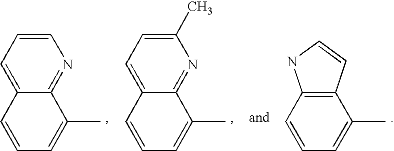

Hal is F, Cl, Br or I , where F and Cl, in particular F, are preferred.

n is preferably 1, 2 or 3, where n equals 3 is particularly preferred.

The substituents R¹, R², A, B and Ar can independently of one another assume one of the abovementioned meanings. The compounds of the general formula I are thus all the more strongly preferred, the more of their substituents have preferred meanings and the greater these meanings are preferred.

Compounds selected from the following group of the compounds Ia to 1h are particularly preferred:

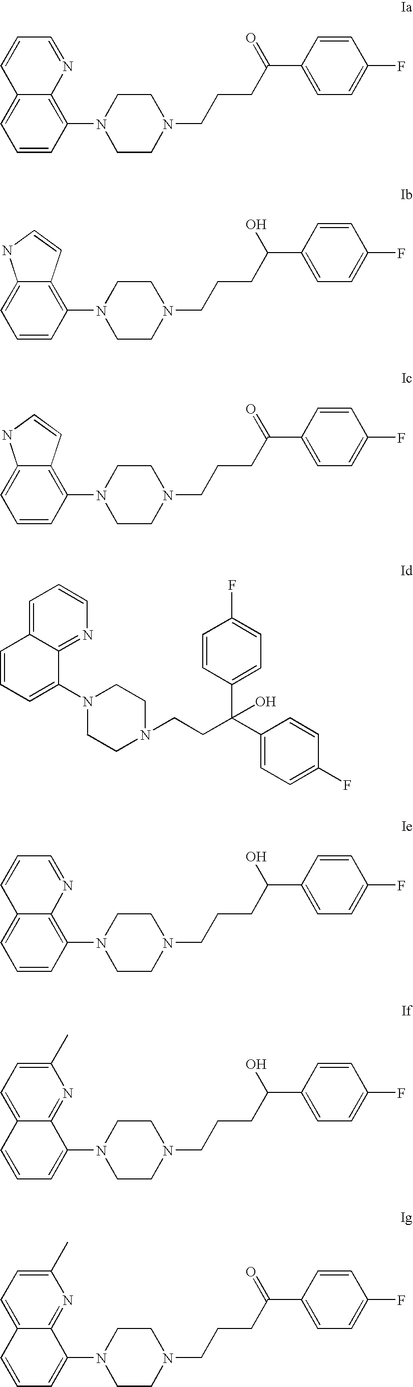

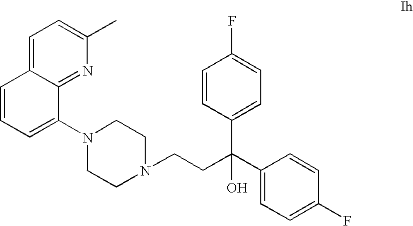

and their salts and solvates.

If the compounds of the general formula I are optically active, the formula I includes both any isolated optical antipodes and the corresponding optionally racemic mixtures in any conceivable composition.

A compound of the general formula I can be converted into the corresponding salt (that is acid addition salt) using an acid. Acids which afford the tolerable (that is biocompatible and adequately bioavailable) salts are suitable for this reaction. It is thus possible to use inorganic acids such as sulfuric acid or hydrohalic acids such as hydrochloric acid, bromic acid or phosphoric acids such as orthophosphoric acid, nitric acid, sulfamic acid, aliphatic, alicyclic, araliphatic, aromatic or heterocyclic monobasic or polybasic carboxylic acids, sulfonic acids or sulfuric acid derivatives such as formic acid, acetic acid, propionic acid, pivalic acid, diethylacetic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methanesulfonic acid or ethanesulfonic acid, ethanedisulfonic acid, 2-hydroxyethanesulfonic acid, benzenesulfonic acid, paratoluenesulfonic acid, naphthalenemonosulfonic acid and naphthalenedisulfonic acid sulfuric acid lauryl ester in order to obtain the corresponding acid addition salt.

If desired, the corresponding free bases of the general formula I can be liberated by the treatment of their salts with strong bases such as sodium hydroxide, potassium hydroxide or sodium or potassium carbonate, provided that no other acidic groups are present in the molecule. In the last-mentioned cases, in which the compounds of the general formula I carry free acidic groups, salt formation can also be brought about by treatment with strong bases. Suitable bases are alkali metal hydroxides, alkaline earth metal hydroxides, or organic bases in the form of primary, secondary or tertiary amines.

Solvates of the compounds of the general formula I are understood as meaning adducts of chemically “inert” solvent molecules to the compounds of the formula I which are formed on account of their mutual attractive force. Solvates are, for example, mono- and dihydrates or addition compounds with alcohols such as methanol or ethanol.

It is known that pharmaceuticals can be converted synthetically into derivatives (for example into alkyl or acyl derivatives, into sugar or oligopeptide derivatives and others) which are converted back into the active compounds of the general formula I in the body metabolically by extracellular or intracellular enzymes. The invention also relates to such “prodrug derivatives” of the compounds of the general formula I.

A further subject of the invention is the use of a compound of the general formula I or of one of its tolerable salts or solvates for the production of a medicament which is suitable for the treatment of human or animal disorders, in particular of disorders of the central nervous system such as pathological stress states, depression and/or psychoses, for the reduction of side effects during the treatment of high blood pressure (e.g. with α-methyldopa), for the, treatment of endocrinological and/or gynaecological disorders, e.g. for the treatment of acromegaly, hypogonadism, secondary amenorrhoea, the post-menstrual syndrome and undesired lactation in puberty and for the prophylaxis and therapy of cerebral disorders (e.g. of migraine), in particular in geriatrics, in a similar manner to specific ergot alkaloids and for the control and prophylaxis of cerebral infarct (cerebral apoplexy) such as cerebral stroke and cerebral ischaemia. Moreover, the pharmaceutical preparations and medicaments which contain a compound of the general formula I are suitable for improvement of the cognitive functional ability and for the treatment of Alzheimer's disease symptoms. In particular, such medicaments are suitable for the treatment of mental disorders of the schizophrenia type and for the control of psychotic anxiety states. The term treatment in the context of the invention includes prophylaxis and therapy of human or animal diseases.

The substances of the general formula I are normally administered analogously to known, commercially obtainable pharmaceutical preparations (e.g. of bromocriptine and dihydroergocornine), preferably in doses of between 0.2 and 500 mg, in particular of between 0.2 and 15 mg per dose unit. The daily dose unit is between 0.001 and 10 mg per kg of body weight. Low doses (of between 0.2 and 1 mg per dose unit, 0.001 to 0.005 mg per kg of body weight) are particularly suitable for pharmaceutical preparations for the treatment of migraine. A dose of between 10 and 50 mg per dose unit is preferred for other indications. However, the dose to be administered depends on a large number of factors, e.g. on the efficacy of the corresponding component, the age, the body weight and the general condition of the patient.

The invention also relates to the compounds of the formula I according to Claim 1 and their physiologically acceptable salts or solvates as pharmaceutical active compounds.

The invention furthermore relates to compounds of the formula I according to Claim 1 and their physiologically acceptable salts or solvates as D₂ receptor antagonists and 5HT_{1A} agonists.

The invention also relates to the compounds of the formula I according to Claim 1 and their physiologically acceptable salts or solvates for use in the control of diseases.

A further subject of the invention is a process for the production of a pharmaceutical preparation, which comprises the conversion of a compound of the general formula I or of one of its tolerable salts or solvates to a suitable dose form together with a suitable vehicle. The compounds of the general formula I can be brought into a suitable dose form together with at least one vehicle or excipient, if appropriate in combination with a further active ingredient.

Suitable vehicles are organic or inorganic substances which are suitable for enteral (e.g. oral) or parenteral or topical administration and which do not react with the substances of the general formula I according to the invention. Examples of such vehicles are water, vegetable oils, benzyl alcohols, polyethylene glycols, gelatin, carbohydrates such as lactose and starch, magnesium stearate, talc and raw petroleum jelly. Tablets, coated tablets, capsules, syrups, juices, drops or suppositories are in particular employed for enteral administration. Solutions, preferably oily or aqueous solutions, such as suspensions, emulsions or alternatively implants are used for parenteral administration. Ointments, creams or powders are employed in the case of external application. The compounds of the general formula I can also be lyophilized and the resulting lyophilizates processed to give injectable preparations.

The invention further relates to medicaments which contain at least one compound of the general formula I or one of its tolerable salts or solvates and, if appropriate, further ingredients such as vehicles, excipients etc. These preparations can be employed as medicaments for the treatment of human or animal diseases.

The aforementioned medicaments can be sterilized and processed together with excipients such as lubricants, preservatives, stabilizers and/or wetting agents, emulsifiers, osmotically active substances, buffers, colorants or flavor enhancers to give other pharmaceutical preparations.

A further subject of the invention is a process for the preparation of compounds of the formula I, and their salts and solvates, characterized in that

(a) a compound of the formula II

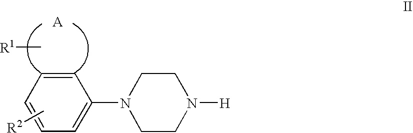

in which R¹, R² and A have the meanings indicated above, is reacted with a compound of the formula III

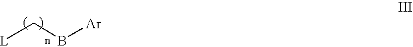

in which Ar, B and n have the meanings indicated above and L is a leaving group, in particular Cl, tosylate or Br, and if B has the meaning —CO— the group B is optionally hydrogenated, alkylated or arylated and, if appropriate, a basic or acidic compound of the formula I is converted into one of its salts or solvates by treating with an acid or base. Grignard or organolithium reagents are preferably used for the alkylation and arylation and a complex hydride is preferably used for the hydrogenation.

The compounds of the formula I and also the starting substances for their preparation are otherwise prepared by methods known per se, such as are described in the literature (e.g. in the standard works such as Houben-Weyl, Methoden der organischen Chemie [Methods of organic chemistry], Georg-Thieme-Verlag, Stuttgart), namely under reaction conditions which are known and suitable for the reactions mentioned. Use can also be made in this case of variants which are known per se, but not mentioned here in greater detail.

If desired, the starting substances can also be formed in situ such that they are not isolated from the reaction mixture, but immediately reacted further to give the compounds of the formula I.

The arylpiperazine derivatives of the formula I are preferably prepared according to the following scheme:

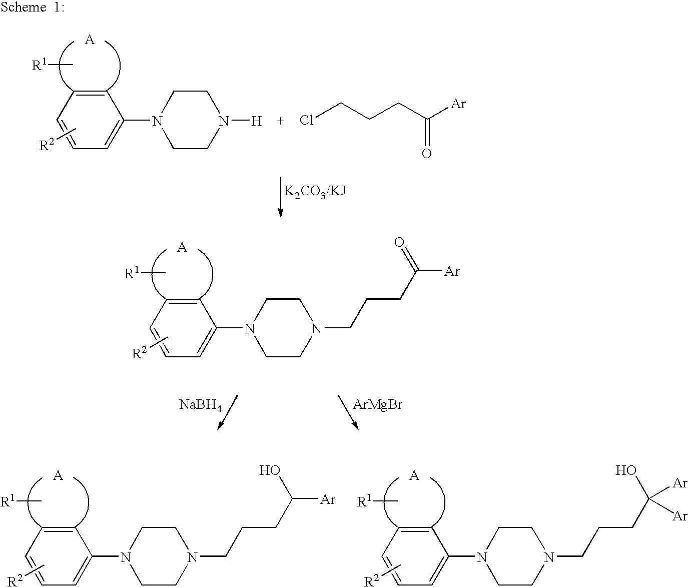

in which A, R¹ and R² have the meanings indicated above.

The invention is described by the following examples.

The molecular weight (M+H⁺) is determined with the aid of electron spray ionization mass spectroscopy. The mass-spectroscopic data derive from HPLC/MS runs (HPLC coupled with an electrospray ionization mass spectrometer). The numerical values are, as customary in this procedure, not the molecular weights of the unmodified compounds, but the molecular weights of the protonated compounds (below: [M+H⁺]). The method is described in the following references: M. Yamashita, J. B. Fenn, J. Phys. Chem. 88, 1984, 4451-4459; C. K. Meng et al., Zeitschrift für Physik D 10, 1988, 361-368; J. B. Fenn et al., Science 246, 1989, 64-71.

### EXAMPLE 1

### 4-[4-(Quinolin-8-yl)piperazin-1-yl]-1-(4-fluorophenyl)butan-1-one trichloride dihydrate

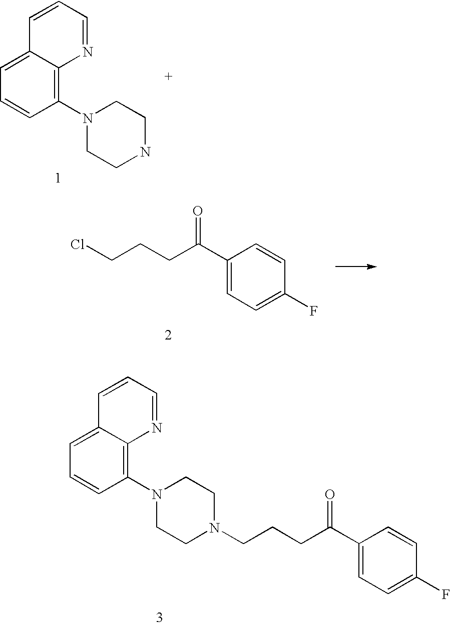

6 g of 1-(Quinolin-8-yl)piperazine 1 and 2.8 g of 4-chloro-1-(4-fluorophenyl)butan-1-one 2 were heated together at 120° (bath temperature) for 1 hour. The mixture was cooled, treated with water and extracted with ethyl acetate. After drying over potassium carbonate, the ethyl acetate was distilled off and the residue was chromatographed on silica gel, whereby 3 was obtained.

For the formation of the acid addition salt, 700 mg of 3 were dissolved in 20 ml of ethyl acetate and acidified with ethanolic HCl. The crystallized hydrochloride was filtered off with suction and washed with ethyl acetate (m.p. 119-120°, [M+H]⁺: 378).

### EXAMPLE 2

### 4-[4-(Quinolin-8-yl)piperazin-1-yl]-1-(4-fluorophenyl)butan-1-ol fumarate

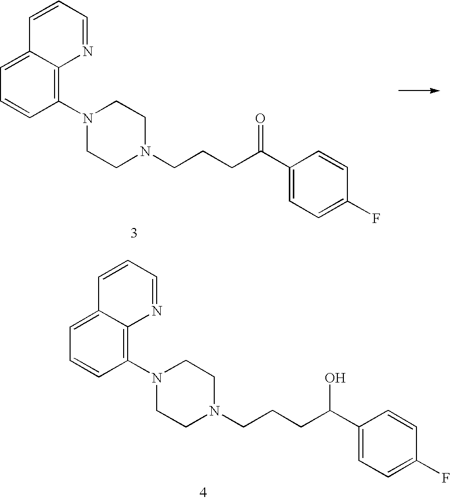

1.3 g of 4-[4-(Quinolin-8-yl)piperazin-1-yl]-1-(4-fluorophenyl)butan-1-one 3 were dissolved in 25 ml of methanol and 264 mg of sodium borohydride were added in portions with stirring and cooling. The mixture was additionally stirred at R.T. for a further two hours, then the methanol was distilled off in vacuo. The residue was treated with water, rendered alkaline with 32% NaOH and extracted with dichloromethane. After drying over potassium carbonate, the dichloromethane was distilled off and the residue was chromatographed on silica gel, whereby 4 was obtained. The residue was dissolved with warming in 20 ml of ethanol with 337 mg of fumaric acid and the solution obtained was evaporated in vacuo. The residue was treated with ethyl acetate, and the crystallized fumarate was filtered off with suction and washed with ethyl acetate (m.p. 145-146°, [M+H]⁺: 380).

### EXAMPLE 3

### 1,1-bis-(4-Fluorophenyl)-4-[4-(2-methylquinolin-8-yl)piperazin-1-yl]-1-butanol fumarate

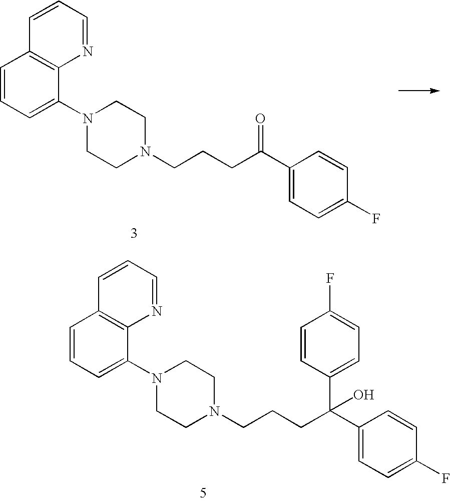

2.2 g of 1-(4-Fluorophenyl)-4-[4-(quinolin-8-yl)piperazin-1-yl]butan-1-one 3 in 20 ml of abs. tetrahydrofuran was added dropwise at R.T. to a Grignard solution of 423 mg of magnesium turnings and 3.05 g of 1-bromo-4-fluorobenzene in 30 ml of abs. tetrahydrofuran. The mixture was stirred overnight at R.T., then 25 ml of 10% ammonium chloride solution were added dropwise with cooling and the mixture was extracted with ethyl acetate. After drying over potassium carbonate, the ethyl acetate was distilled off and the residue was chromatographed on silica gel, whereby 5 was obtained. The residue was dissolved with warming in 30 ml of ethanol with 290 mg of fumaric acid. The solution was cooled, and the crystallized fumarate was filtered off with suction and washed with ethanol and ethyl acetate (m.p. 219-220°, M⁺: 473).

### EXAMPLE 4

### 1-(4-Fluorophenyl)-4-[4-(2-methylquinolin-8-yl)piperazin-1-yl]butan-1-one hemifumarate

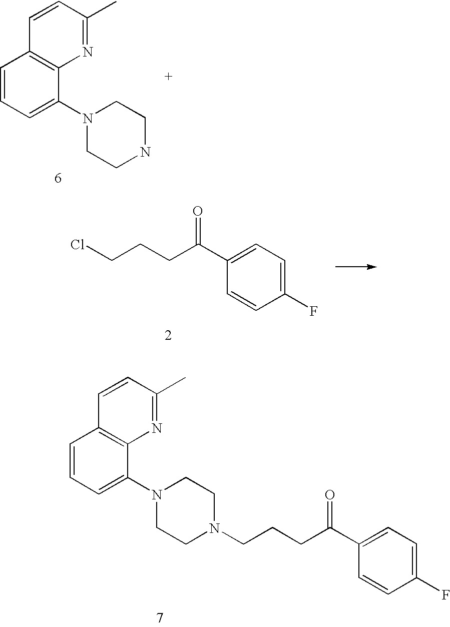

3.53 g of 4-chloro-1-(4-fluorophenyl)butan-1-one 2 were added to 4 g of 1-(2-methylquinolin-8-yl)piperazine 6, 2.43 g of potassium carbonate and 20 mg of potassium iodide in 60 ml of acetonitrile and the mixture was stirred at 80° for 87 hours in a heating block. The acetonitrile was then distilled off in vacuo, and the residue was treated with water and extracted with dichloromethane. After drying over potassium carbonate, the dichloromethane was distilled off and the residue was chromatographed on silica gel, whereby 7 was obtained.

For the formation of the acid addition salts, 1.2 g of 7 were dissolved with warming in 15 ml of ethanol with 348 mg of fumaric acid. The fumarate which crystallized on cooling was filtered off with suction and washed with ethanol (m.p. 195-196°, [M+H]⁺: 392).

### EXAMPLE 5

### 4-[4-(2-Methylquinolin-8-yl)piperazin-1-yl]-1-(4-fluorophenyl)butan-1-ol sesquifumarate

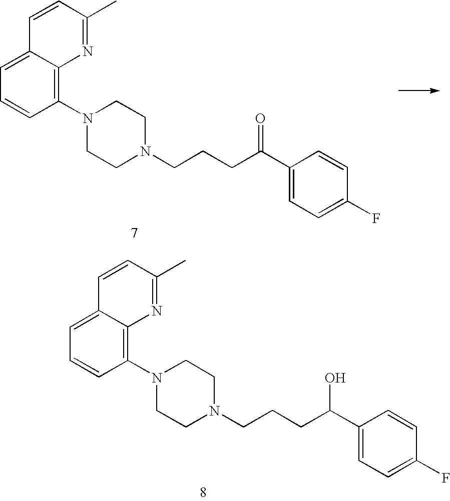

Analogously to Example 2, using

| | | |
|---|---|---|
| 1.3 | g (0.0033 mol) | of 4-[4-(2-methylquinolin-8-yl)piperazin-1-yl]- |
| | | 1-(4-fluorophenyl)butan-1-one 7, |
| 249 | mg (0.0066 mol) | of sodium borohydride and |
| 25 | ml | of methanol, the compound 8 was obtained. |

For the formation of the acid addition salts, 830 mg of 8 were dissolved with warming in 10 ml of ethanol with 244 mg of fumaric acid and the solution was evaporated in vacuo. The residue was treated with ethyl acetate and the crystal obtained were filtered off with suction and washed with ethyl acetate (m.p. 164-165°, [M+H]⁺: 394).

### EXAMPLE 6

### 1,1-bis(4-Fluorophenyl)-4-[4-(2-methylquinolin-8-yl)-1-butanol hemifumarate ethanoate

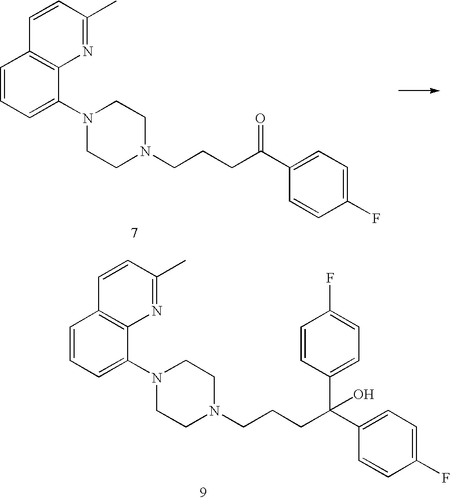

Analogously to Example 3, using

| | | |
|---|---|---|
| 539 | mg | of magnesium turnings, |
| 3.9 | g (0.022 mol) | of 1-bromo-4-fluorobenzene, |
| 2.9 | g (0.007 mol) | of 1-(4-fluorophenyl)-4-[4-(2-methylquinolin-8- |
| | | yl) piperazin-1-yl]butan-1-one 7 and |
| 50 | ml | of abs. tetrahydrofuran |

the compound 9 was obtained.

For the formation of the acid addition salt, 2.3 g 9 were dissolved with warming in 20 ml of ethanol with 545 mg of fumaric acid. The fumarate which crystallized after cooling was filtered off with suction and washed with ethyl acetate (m.p. 129-130°, [M+H]⁺: 488).

### EXAMPLE 7

### 4-[4-(Indol-4-yl)piperazin-1-yl]-1-(4-fluorophenyl)butan-1-one dihydrochloride

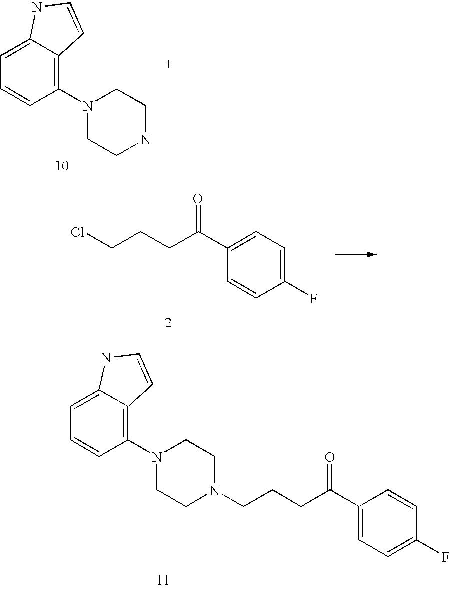

Analogously to Example 4, using

| | | |
|---|---|---|
| 4 | g (0.02 mol) | of 1-(indol-4-yl)piperazine 10, |
| 4 | g (0.02 mol) | of 4-chloro-1-(4-fluorophenyl)butan-1-one 2, |
| 2.8 | g (0.02 mol) | of potassium carbonate, |
| 40 | mg | of potassium iodide and |
| 75 | ml | of acetonitrile, the compound 11 was obtained. |

For the formation of the acid addition salt, 800 mg of base were dissolved in 10 ml of ethanol with warming and acidified with ethanol/HCl. The hydrochloride which crystallized after cooling was filtered off with suction and washed with ethanol and ether (m.p. 233-234°, [M+H]⁺: 366).

### EXAMPLE 8

### 4-[4-(Indol-4-yl)piperazin-1-yl]-1-(4-fluorophenyl)-1-butanol dihydrochloride

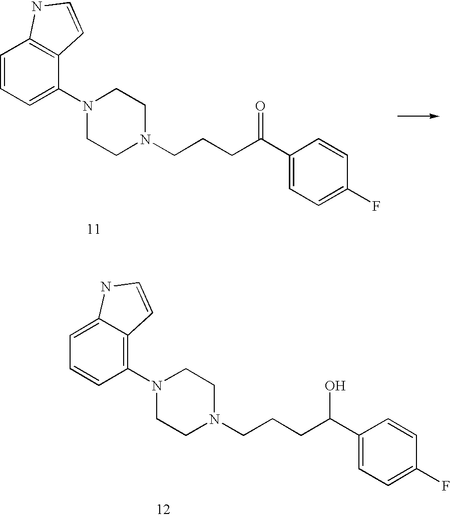

Analogously to Example 2, using

| | | |
|---|---|---|
| 1.29 | (0.0033 mol) | of 4-[4-(indol-4-yl)piperazin-1-yl]-1-(4-fluoro- |
| | | phenyl)butan-1-one 11, |
| 250 | mg (0.0066 mol) | of sodium borohydride and a mixture of |
| 30 | ml | of methanol and |
| 20 | ml | of dichloromethane, the compound 12 was |
| | | obtained. |

For the formation of the acid addition salt, 1.1 g of 12 were dissolved in ethanol with warming and acidified with ethanolic HCl. The hydrochloride which crystallized after cooling was filtered off with suction and washed with ethanol and ether (m.p. 227-22820 , [M+H]⁺: 368).

The following compounds and their acid addition salts are prepared analogously using the appropriate precursors.

### EXAMPLES 9-76

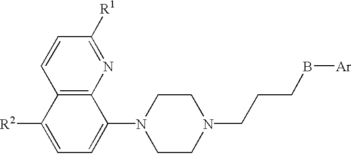

| | R¹ | R² | B | Ar |
|---|---|---|---|---|
| (9) | H | H | —CO— | p-C₆H₄CN |
| (10) | H | H | —CO— | o-C₆H₄F |
| (11) | H | H | —CO— | m-C₆H₄F |
| (12) | H | H | —CO— | p-C₆H₄Cl |
| (13) | H | H | —CO— | m-C₆H₄Cl |
| (14) | H | H | —CO— | C₆H₅ |
| (15) | H | H | —CO— | 2-C₄H₃S |
| (16) | H | H | —CH(OH)— | p-C₅H₄CN |
| (17) | H | H | —CH(OH)— | o-C₆H₄F |
| (18) | H | H | —CH(OH)— | m-C₆H₄F |
| (19) | H | H | —CH(OH)— | p-C₆H₄Cl |
| (20) | H | H | —CH(OH)— | m-C₆H₄Cl |
| (21) | H | H | —CH(OH)— | C₆H₅ |
| (22) | H | H | —CH(OH)— | 2-C₄H₃S |
| (23) | H | H | —C(p-C₆H₄F)(OH)— | p-C₆H₄CN |
| (24) | H | H | —C(p-C₆H₄F)(OH)— | o-C₆H₄F |
| (25) | H | H | —C(p-C₆H₄F)(OH)— | m-C₆H₄F |
| (26) | H | H | —C(p-C₆H₄F)(OH)— | p-C₆H₄Cl |
| (27) | H | H | —C(p-C₆H₄F)(OH)— | m-C₆H₄Cl |
| (28) | H | H | —C(p-C₆H₄F)(OH)— | C₆H₅ |
| (29) | H | H | —C(p-C₆H₄F)(OH)— | 2-C₄H₃S |
| (30) | H | H | —C(C₆H₅)(OH)— | p-C₆H₄F |
| (31) | H | H | —C(C₆H₅)(OH)— | o-C₆H₄F |
| (32) | H | H | —C(C₆H₅)(OH)— | m-C₆H₄F |
| (33) | H | H | —C(C₆H₅)(OH)— | p-C₆H₄Cl |
| (34) | H | H | —C(C₆H₅)(OH)— | m-C₆H₄Cl |
| (35) | H | H | —C(C₆H₅)(OH)— | C₆H₅ |
| (36) | H | H | —C(C₆H₅)(OH)— | 2-C₄H₃S |
| (37) | H | CH₃ | —CO— | p-C₆H₄F |
| (38) | H | CH₃ | —CO— | o-C₆H₄F |
| (39) | H | CH₃ | —CO— | m-C₆H₄F |
| (40) | H | CH₃ | —CO— | p-C₆H₄F |
| (41) | H | CH₃ | —CO— | m-C₆H₄F |
| (42) | H | CH₃ | —CO— | C₆H₅ |
| (43) | H | CH₃ | —CO— | 2-C₄H₃S |
| (44) | H | CH₃ | —CH(OH)— | p-C₆H₄F |
| (45) | H | CH₃ | —CH(OH)— | o-C₆H₄F |
| (46) | H | CH₃ | —CH(OH)— | m-C₆H₄F |
| (47) | H | CH₃ | —CH(OH)— | p-C₆H₄Cl |
| (48) | H | CH₃ | —CH(OH)— | m-C₆H₄Cl |
| (49) | H | CH₃ | —CH(OH)— | C₆H₅ |
| (50) | H | CH₃ | —CH(OH)— | 2-C₄H₃S |
| (51) | H | CH₃ | —C(p-C₆H₄F)(OH)— | p-C₆H₄F |
| (52) | H | CH₃ | —C(p-C₆H₄F)(OH)— | o-C₆H₄F |
| (53) | H | CH₃ | —C(p-C₆H₄F)(OH)— | m-C₆H₄F |
| (54) | H | CH₃ | —C(p-C₆H₄F)(OH)— | p-C₆H₄Cl |
| (55) | H | CH₃ | —C(p-C₆H₄F)(OH)— | m-C₆H₄Cl |
| (56) | H | CH₃ | —C(p-C₆H₄F)(OH)— | C₆H₅ |
| (57) | H | CH₃ | —C(p-C₆H₄F)(OH)— | 2-C₄H₃S |
| (58) | H | CH₃ | —C(C₆H₅)(OH)— | p-C₆H₄F |
| (59) | H | CH₃ | —C(C₆H₅)(OH)— | o-C₆H₄F |
| (60) | H | CH₃ | —C(C₆H₅)(OH)— | m-C₆H₄F |
| (61) | H | CH₃ | —C(C₆H₅)(OH)— | p-C₆H₄Cl |
| (62) | H | CH₃ | —C(C₆H₅)(OH)— | m-C₆H₄Cl |
| (63) | H | CH₃ | —C(C₆H₅)(OH)— | C₆H₅ |
| (64) | H | CH₃ | —C(C₆H₅)(OH)— | 2-C₄H₃S |
| (65) | CH₃ | H | —CO— | p-C₆H₄CN |
| (66) | CH₃ | H | —C(C₆H₅)(OH)— | p-C₆H₄F |
| (67) | CH₃ | H | —C(C₆H₅)(OH)— | p-C₆H₄CN |
| (68) | CH₃ | H | —CH(OH)— | p-C₆H₄CN |
| (69) | H | F | —CO— | p-C₆H₄F |
| (70) | H | F | —C(C₆H₅)(OH)— | p-C₆H₄F |
| (71) | H | F | —C(p-C₆H₄F)(OH)— | p-C₆H₄F |
| (72) | H | Cl | —CH(OH)— | p-C₆H₄F |
| (73) | F | CH₃ | —CO— | p-C₆H₄F |
| (74) | F | CH₃ | —C(C₆H₅)(OH)— | p-C₆H₄F |
| (75) | F | CH₃ | —C(p-C₆H₄F)(OH)— | p-C₆H₄F |
| (76) | Cl | CH₃ | —CH(OH)— | p-C₆H₄F |

### EXAMPLES 77-144

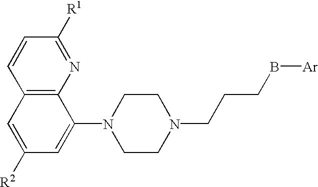

| | R¹ | R² | B | Ar |
|---|---|---|---|---|
| (77) | H | Cl | —CO— | p-C₆H₄F |
| (78) | H | Cl | —CO— | o-C₆H₄F |
| (79) | H | Cl | —CO— | m-C₆H₄F |
| (80) | H | Cl | —CO— | p-C₆H₄Cl |
| (81) | H | Cl | —CO— | m-C₆H₄Cl |
| (82) | H | Cl | —CO— | C₆H₅ |
| (83) | H | Cl | —CO— | 2-C₄H₃S |
| (84) | H | Cl | —CH(OH)— | p-C₆H₄F |
| (85) | H | Cl | —CH(OH)— | o-C₆H₄F |
| (86) | H | Cl | —CH(OH)— | m-C₆H₄F |
| (87) | H | Cl | —CH(OH)— | p-C₆H₄Cl |
| (88) | H | Cl | —CH(OH)— | m-C₆H₄Cl |
| (89) | H | Cl | —CH(OH)— | C₆H₅ |
| (90) | H | Cl | —CH(OH)— | 2-C₄H₃S |
| (91) | H | Cl | —C(p-C₆H₄F)(OH)— | p-C₆H₄F |
| (92) | H | Cl | —C(p-C₆H₄F)(OH)— | o-C₆H₄F |
| (93) | H | Cl | —C(p-C₆H₄F)(OH)— | m-C₆H₄F |
| (94) | H | Cl | —C(p-C₆H₄F)(OH)— | p-C₆H₄Cl |
| (95) | H | Cl | —C(p-C₆H₄F)(OH)— | m-C₆H₄Cl |
| (96) | H | Cl | —C(p-C₆H₄F)(OH)— | C₆H₅ |
| (97) | H | Cl | —C(p-C₆H₄F)(OH)— | 2-C₄H₃S |
| (98) | H | Cl | —C(C₆H₅)(OH)— | p-C₆H₄F |
| (99) | H | F | —C(C₆H₅)(OH)— | o-C₆H₄F |
| (100) | H | F | —C(C₆H₅)(OH)— | m-C₆H₄F |
| (101) | H | F | —C(C₆H₅)(OH)— | p-C₆H₄Cl |
| (102) | H | F | —C(C₆H₅)(OH)— | m-C₆H₄Cl |
| (103) | H | F | —C(C₆H₅)(OH)— | C₆H₅ |
| (104) | H | F | —C(C₆H₅)(OH)— | 2-C₄H₃S |
| (105) | H | CH₃ | —CO— | p-C₆H₄F |
| (106) | H | CH₃ | —CO— | o-C₆H₄F |
| (107) | H | CH₃ | —CO— | m-C₆H₄F |
| (108) | H | CH₃ | —CO— | p-C₆H₄Cl |
| (109) | H | CH₃ | —CO— | m-C₆H₄Cl |
| (110) | H | CH₃ | —CO— | C₆H₅ |
| (111) | H | CH₃ | —CO— | 2-C₄H₃S |
| (112) | H | CH₃ | —CH(OH)— | p-C₆H₄F |
| (113) | H | CH₃ | —CH(OH)— | o-C₆H₄F |
| (114) | H | CH₃ | —CH(OH)— | m-C₆H₄F |
| (115) | H | CH₃ | —CH(OH)— | p-C₆H₄Cl |
| (116) | H | CH₃ | —CH(OH)— | m-C₆H₄Cl |
| (117) | H | CH₃ | —CH(OH)— | C₆H₅ |
| (118) | H | CH₃ | —CH(OH)— | 2-C₄H₃S |
| (119) | H | CH₃ | —C(p-C₆H₄F)(OH)— | p-C₆H₄F |
| (120) | H | CH₃ | —C(p-C₆H₄F)(OH)— | o-C₆H₄F |
| (121) | H | CH₃ | —C(p-C₆H₄F)(OH)— | m-C₆H₄F |
| (122) | H | CH₃ | —C(p-C₆H₄F)(OH)— | p-C₆H₄Cl |
| (123) | H | CH₃ | —C(p-C₆H₄F)(OH)— | m-C₆H₄Cl |
| (124) | H | CH₃ | —C(p-C₆H₄F)(OH)— | C₆H₅ |
| (125) | H | CH₃ | —C(p-C₆H₄F)(OH)— | 2-C₄H₃S |
| (126) | H | CH₃ | —C(C₆H₅)(OH)— | p-C₆H₄F |
| (127) | H | CH₃ | —C(C₆H₅)(OH)— | o-C₆H₄F |
| (128) | H | CH₃ | —C(C₆H₅)(OH)— | m-C₆H₄F |
| (129) | H | CH₃ | —C(C₆H₅)(OH)— | p-C₆H₄Cl |
| (130) | H | CH₃ | —C(C₆H₅)(OH)— | m-C₆H₄Cl |
| (131) | H | CH₃ | —C(C₆H₅)(OH)— | C₆H₅ |
| (132) | H | CH₃ | —C(C₆H₅)(OH)— | 2-C₄H₃S |
| (133) | CH₃ | Cl | —CO— | p-C₆H₄F |
| (134) | CH₃ | Cl | —C(C₆H₅)(OH)— | p-C₆H₄F |
| (135) | CH₃ | Cl | —C(C₆H₄F)(OH)— | p-C₆H₄F |
| (136) | CH₃ | Cl | —CH(OH)— | p-C₆H₄F |
| (137) | H | F | —CO— | p-C₆H₄F |
| (138) | H | F | —C(C₆H₅)(OH)— | p-C₆H₄F |
| (139) | H | F | —C(p-C₆H₄F)(OH)— | p-C₆H₄F |
| (140) | H | Cl | —CH(OH)— | p-C₆H₄F |
| (141) | F | CH₃ | —OH— | p-C₆H₄F |
| (142) | F | CH₃ | —C(C₆H₅)(OH)— | p-C₆H₄F |
| (143) | F | CH₃ | —C(p-C₆H₄F)(OH)— | p-C₆H₄F |
| (144) | Cl | CH₃ | —CH(OH)— | p-C₆H₄F |

### EXAMPLES 145-212

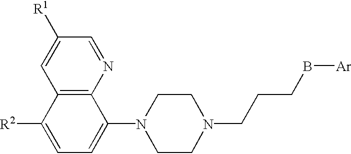

| | R¹ | R² | B | Ar |
|---|---|---|---|---|
| (145) | CH₃ | H | —CO— | p-C₆H₄F |
| (146) | CH₃ | H | —CO— | o-C₆H₄F |
| (147) | CH₃ | H | —CO— | m-C₆H₄F |
| (148) | CH₃ | H | —CO— | p-C₆H₄Cl |
| (149) | CH₃ | H | —CO— | m-C₆H₄Cl |
| (150) | CH₃ | H | —CO— | C₆H₅ |
| (151) | CH₃ | H | —CO— | 2-C₄H₃S |
| (152) | CH₃ | H | —CH(OH)— | p-C₆H₄F |
| (153) | CH₃ | H | —CH(OH)— | o-C₆H₄F |
| (154) | CH₃ | H | —CH(OH)— | m-C₆H₄F |
| (155) | CH₃ | H | —CH(OH)— | p-C₆H₄Cl |
| (156) | CH₃ | H | —CH(OH)— | m-C₆H₄Cl |
| (157) | CH₃ | H | —CH(OH)— | C₆H₅ |
| (158) | CH₃ | H | —CH(OH)— | 2-C₄H₃S |
| (159) | CH₃ | H | —C(p-C₅H₄F)(OH)— | p-C₆H₄F |
| (160) | CH₃ | H | —C(p-C₅H₄F)(OH)— | o-C₆H₄F |
| (161) | CH₃ | H | —C(p-C₅H₄F)(OH)— | m-C₆H₄F |
| (162) | CH₃ | H | —C(p-C₅H₄F)(OH)— | p-C₆H₄Cl |
| (163) | CH₃ | H | —C(p-C₅H₄F)(OH)— | m-C₆H₄Cl |
| (164) | CH₃ | H | —C(p-C₅H₄F)(OH)— | C₆H₅ |
| (165) | CH₃ | H | —C(p-C₅H₄F)(OH)— | 2-C₄H₃S |
| (166) | CH₃ | H | —C(C₆H₅)(OH)— | p-C₆H₄F |
| (167) | CH₃ | H | —C(C₆H₅)(OH)— | o-C₆H₄F |
| (168) | CH₃ | H | —C(C₆H₅)(OH)— | m-C₆H₄F |
| (169) | CH₃ | H | —C(C₆H₅)(OH)— | p-C₆H₄Cl |
| (170) | CH₃ | H | —C(C₆H₅)(OH)— | m-C₆H₄Cl |
| (171) | CH₃ | H | —C(C₆H₅)(OH)— | C₆H₅ |
| (172) | CH₃ | H | —C(C₆H₅)(OH)— | 2-C₄H₃S |
| (173) | CH₃ | CH₃ | —CO— | p-C₆H₄F |
| (174) | CH₃ | CH₃ | —CO— | o-C₆H₄F |
| (175) | CH₃ | CH₃ | —CO— | m-C₆H₄F |
| (176) | CH₃ | CH₃ | —CO— | p-C₆H₄Cl |
| (177) | CH₃ | CH₃ | —CO— | m-C₆H₄Cl |
| (178) | CH₃ | CH₃ | —CO— | C₆H₅ |
| (179) | CH₃ | CH₃ | —CO— | 2-C₄H₃S |
| (180) | CH₃ | CH₃ | —CH(OH)— | p-C₆H₄F |
| (181) | CH₃ | CH₃ | —CH(OH)— | o-C₆H₄F |
| (182) | CH₃ | CH₃ | —CH(OH)— | m-C₆H₄F |
| (183) | CH₃ | CH₃ | —CH(OH)— | p-C₆H₄Cl |
| (184) | CH₃ | CH₃ | —CH(OH)— | m-C₆H₄Cl |
| (185) | CH₃ | CH₃ | —CH(OH)— | C₆H₅ |
| (186) | CH₃ | CH₃ | —CH(OH)— | 2-C₄H₃S |
| (187) | CH₃ | CH₃ | —C(p-C₆H₄F)(OH)— | p-C₆H₄F |
| (188) | CH₃ | CH₃ | —C(p-C₆H₄F)(OH)— | o-C₆H₄F |
| (189) | CH₃ | CH₃ | —C(p-C₆H₄F)(OH)— | m-C₆H₄F |
| (190) | CH₃ | CH₃ | —C(p-C₆H₄F)(OH)— | p-C₆H₄Cl |
| (191) | CH₃ | CH₃ | —C(p-C₆H₄F)(OH)— | m-C₆H₄Cl |
| (192) | CH₃ | CH₃ | —C(p-C₆H₄F)(OH)— | C₆H₅ |
| (193) | CH₃ | CH₃ | —C(p-C₆H₄F)(OH)— | 2-C₄H₃S |
| (194) | CH₃ | CH₃ | —C(C₆H₅)(OH)— | p-C₆H₄F |
| (195) | CH₃ | CH₃ | —C(C₆H₅)(OH)— | o-C₆H₄F |
| (196) | CH₃ | CH₃ | —C(C₆H₅)(OH)— | m-C₆H₄F |
| (197) | CH₃ | CH₃ | —C(C₆H₅)(OH)— | p-C₆H₄Cl |
| (198) | CH₃ | CH₃ | —C(C₆H₅)(OH)— | m-C₆H₄Cl |
| (199) | CH₃ | CH₃ | —C(C₆H₅)(OH)— | C₆H₅ |
| (200) | CH₃ | CH₃ | —C(C₆H₅)(OH)— | 2-C₄H₃S |
| (201) | CH₃ | H | —CO— | p-C₆H₄F |
| (202) | CH₃ | H | —C(C₆H₅)(OH)— | p-C₆H₄F |
| (203) | CH₃ | H | —C(C₆H₅)(OH)— | p-C₆H₄F |
| (204) | CH₃ | H | —CH(OH)— | p-C₆H₄F |
| (205) | Cl | F | —CO— | p-C₆H₄F |
| (206) | Cl | F | —C(C₆H₅)(OH)— | p-C₆H₄F |
| (207) | F | F | —C(p-C₆H₄F)(OH)— | p-C₆H₄F |
| (208) | F | Cl | —CH(OH)— | p-C₆H₄F |
| (209) | F | CH₃ | —CO— | p-C₆H₄F |
| (210) | F | CH₃ | —C(C₆H₅)(OH)— | p-C₆H₄F |
| (211) | F | CH₃ | —C(p-C₆H₄F)(OH)— | p-C₆H₄F |
| (212) | Cl | CH₃ | —CH(OH)— | p-C₆H₄F |

### EXAMPLES 213-280

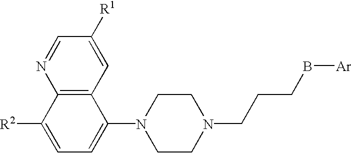

| | R¹ | R² | B | Ar |
|---|---|---|---|---|
| (213) | H | H | —CO— | p-C₆H₄F |
| (214) | H | H | —CO— | o-C₆H₄F |
| (215) | H | H | —CO— | m-C₆H₄F |
| (216) | H | H | —CO— | p-C₆H₄Cl |
| (217) | H | H | —CO— | m-C₆H₄Cl |
| (218) | H | H | —CO— | C₆H₅ |
| (219) | H | H | —CO— | 2-C₄H₃S |
| (220) | H | H | —CH(OH)— | p-C₆H₄F |
| (221) | H | H | —CH(OH)— | o-C₆H₄F |
| (222) | H | H | —CH(OH)— | m-C₆H₄F |
| (223) | H | H | —CH(OH)— | p-C₆H₄Cl |
| (224) | H | H | —CH(OH)— | m-C₆H₄Cl |
| (225) | H | H | —CH(OH)— | C₆H₅ |
| (226) | H | H | —CH(OH)— | 2-C₄H₃S |
| (227) | H | H | —C(p-C₆H₄F)(OH)— | p-C₆H₄F |
| (228) | H | H | —C(p-C₆H₄F)(OH)— | o-C₆H₄F |
| (229) | H | H | —C(p-C₆H₄F)(OH)— | m-C₆H₄F |
| (230) | H | H | —C(p-C₆H₄F)(OH)— | p-C₆H₄Cl |
| (231) | H | H | —C(p-C₆H₄F)(OH)— | m-C₆H₄Cl |
| (232) | H | H | —C(p-C₆H₄F)(OH)— | C₆H₅ |
| (233) | H | H | —C(p-C₆H₄F)(OH)— | 2-C₄H₃S |
| (234) | H | H | —C(C₆H₅)(OH)— | p-C₆H₄F |
| (235) | H | H | —C(C₆H₅)(OH)— | o-C₆H₄F |
| (236) | H | H | —C(C₆H₅)(OH)— | m-C₆H₄F |
| (237) | H | H | —C(C₆H₅)(OH)— | p-C₆H₄Cl |
| (238) | H | H | —C(C₆H₅)(OH)— | m-C₆H₄Cl |
| (239) | H | H | —C(C₆H₅)(OH)— | C₆H₅ |
| (240) | H | H | —C(C₆H₅)(OH)— | 2-C₄H₃S |
| (241) | H | CH₃ | —CO— | p-C₆H₄F |
| (242) | H | CH₃ | —CO— | o-C₆H₄F |
| (243) | H | CH₃ | —CO— | m-C₆H₄F |
| (244) | H | CH₃ | —CO— | p-C₆H₄Cl |
| (245) | H | CH₃ | —CO— | m-C₆H₄Cl |
| (246) | H | CH₃ | —CO— | C₆H₅ |
| (247) | H | CH₃ | —CO— | 2-C₄H₃S |
| (248) | H | CH₃ | —CH(OH)— | p-C₆H₄F |
| (249) | H | CH₃ | —CH(OH)— | o-C₆H₄F |
| (250) | H | CH₃ | —CH(OH)— | m-C₆H₄F |
| (251) | H | CH₃ | —CH(OH)— | p-C₆H₄Cl |
| (252) | H | CH₃ | —CH(OH)— | m-C₆H₄Cl |
| (253) | H | CH₃ | —CH(OH)— | C₆H₅ |
| (254) | H | CH₃ | —CH(OH)— | 2-C₄H₃S |
| (255) | H | CH₃ | —C(p-C₆H₄F)(OH)— | p-C₆H₄F |
| (256) | H | CH₃ | —C(p-C₆H₄F)(OH)— | o-C₆H₄F |
| (257) | H | CH₃ | —C(p-C₆H₄F)(OH)— | m-C₆H₄F |
| (258) | H | CH₃ | —C(p-C₆H₄F)(OH)— | p-C₆H₄Cl |
| (259) | H | CH₃ | —C(p-C₆H₄F)(OH)— | m-C₆H₄Cl |
| (260) | H | CH₃ | —C(p-C₆H₄F)(OH)— | C₆H₅ |
| (261) | H | CH₃ | —C(p-C₆H₄F)(OH)— | 2-C₄H₃S |
| (262) | H | CH₃ | —C(C₆H₅)(OH)— | p-C₆H₄F |
| (263) | H | CH₃ | —C(C₆H₅)(OH)— | o-C₆H₄F |
| (264) | H | CH₃ | —C(C₆H₅)(OH)— | m-C₆H₄F |
| (265) | H | CH₃ | —C(C₆H₅)(OH)— | p-C₆H₄Cl |
| (266) | H | CH₃ | —C(C₆H₅)(OH)— | m-C₆H₄Cl |
| (267) | H | CH₃ | —C(C₆H₅)(OH)— | C₆H₅ |
| (268) | H | CH₃ | —C(C₆H₅)(OH)— | 2-C₄H₃S |
| (269) | CH₃ | H | —CO— | p-C₆H₄F |
| (270) | CH₃ | H | —C(C₆H₅)(OH)— | p-C₆H₄F |
| (271) | CH₃ | H | —C(C₆H₅)(OH)— | p-C₆H₄CN |
| (272) | CH₃ | H | —CH(OH)— | p-C₆H₄F |
| (273) | H | F | —CO— | p-C₆H₄F |
| (274) | H | F | —C(C₆H₅)(OH)— | p-C₆H₄F |
| (275) | H | F | —C(p-C₆H₄F)(OH)— | p-C₆H₄F |
| (276) | H | Cl | —CH(OH)— | p-C₆H₄F |
| (277) | F | CH₃ | —CO— | p-C₆H₄F |
| (278) | F | CH₃ | —C(C₆H₅)(OH)— | p-C₆H₄F |
| (279) | F | CH₃ | —C(p-C₆H₄F)(OH)— | p-C₆H₄F |
| (280) | Cl | CH₃ | —CH(OH)— | p-C₆H₄F |

### EXAMPLES 281-348

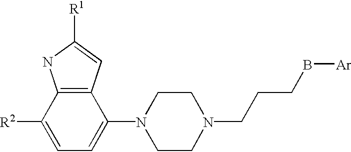

| | R¹ | R² | B | Ar |
|---|---|---|---|---|
| (281) | H | H | —CO— | p-C₆H₄CN |
| (282) | H | H | —CO— | o-C₆H₄F |
| (283) | H | H | —CO— | m-C₆H₄F |
| (284) | H | H | —CO— | p-C₆H₄Cl |
| (255) | H | H | —CO— | m-C₆H₄Cl |
| (286) | H | H | —CO— | C₆H₅ |
| (287) | H | H | —CO— | 2-C₄H₃S |
| (288) | H | H | —CH(OH)— | p-C₆H₄CN |
| (289) | H | H | —CH(OH)— | o-C₆H₄F |
| (290) | H | H | —CH(OH)— | m-C₆H₄F |
| (291) | H | H | —CH(OH)— | p-C₆H₄Cl |
| (292) | H | H | —CH(OH)— | m-C₆H₄Cl |
| (293) | H | H | —CH(OH)— | C₆H₅ |
| (294) | H | H | —CH(OH)— | 2-C₄H₃S |
| (295) | H | H | —C(p-C₆H₄F)(OH)— | p-C₆H₄F |
| (296) | H | H | —C(p-C₆H₄F)(OH)— | o-C₆H₄F |
| (297) | H | H | —C(p-C₆H₄F)(OH)— | m-C₆H₄F |
| (298) | H | H | —C(p-C₆H₄F)(OH)— | p-C₆H₄Cl |
| (299) | H | H | —C(p-C₆H₄F)(OH)— | m-C₆H₄Cl |
| (300) | H | H | —C(p-C₆H₄F)(OH)— | C₆H₅ |
| (301) | H | H | —C(p-C₆H₄F)(OH)— | 2-C₄H₃S |
| (302) | H | H | —C(C₆H₅)(OH)— | p-C₆H₄CN |
| (303) | H | H | —C(C₆H₅)(OH)— | o-C₆H₄F |
| (304) | H | H | —C(C₆H₅)(OH)— | m-C₆H₄F |
| (305) | H | H | —C(C₆H₅)(OH)— | p-C₆H₄Cl |
| (306) | H | H | —C(C₆H₅)(OH)— | m-C₆H₄Cl |
| (307) | H | H | —C(C₆H₅)(OH)— | C₆H₅ |
| (308) | H | H | —C(C₆H₅)(OH)— | 2-C₄H₃S |
| (309) | H | CH₃ | —CO— | p-C₆H₄F |
| (310) | H | CH₃ | —CO— | o-C₆H₄F |
| (311) | H | CH₃ | —CO— | m-C₆H₄F |
| (312) | H | CH₃ | —CO— | p-C₆H₄Cl |
| (313) | H | CH₃ | —CO— | m-C₆H₄Cl |
| (314) | H | CH₃ | —CO— | C₆H₅ |
| (315) | H | CH₃ | —CO— | 2-C₄H₃S |
| (316) | H | CH₃ | —CH(OH)— | p-C₆H₄F |
| (317) | H | CH₃ | —CH(OH)— | o-C₆H₄F |
| (318) | H | CH₃ | —CH(OH)— | m-C₆H₄F |
| (319) | H | CH₃ | —CH(OH)— | p-C₆H₄Cl |
| (320) | H | CH₃ | —CH(OH)— | m-C₆H₄Cl |
| (321) | H | CH₃ | —CH(OH)— | C₆H₅ |
| (322) | H | CH₃ | —CH(OH)— | 2-C₄H₃S |
| (323) | H | CH₃ | —C(p-C₆H₄F)(OH)— | p-C₆H₄F |
| (324) | H | CH₃ | —C(p-C₆H₄F)(OH)— | o-C₆H₄F |
| (325) | H | CH₃ | —C(p-C₆H₄F)(OH)— | m-C₆H₄F |
| (326) | H | CH₃ | —C(p-C₆H₄F)(OH)— | p-C₆H₄Cl |
| (327) | H | CH₃ | —C(p-C₆H₄F)(OH)— | m-C₆H₄Cl |
| (328) | H | CH₃ | —C(p-C₆H₄F)(OH)— | C₆H₅ |
| (329) | H | CH₃ | —C(p-C₆H₄F)(OH)— | 2-C₄H₃S |
| (330) | H | CH₃ | —C(C₆H₅)(OH)— | p-C₆H₄F |
| (331) | H | CH₃ | —C(C₆H₅)(OH)— | o-C₆H₄F |
| (332) | H | CH₃ | —C(C₆H₅)(OH)— | m-C₆H₄F |
| (333) | H | CH₃ | —C(C₆H₅)(OH)— | p-C₆H₄Cl |
| (334) | H | CH₃ | —C(C₆H₅)(OH)— | m-C₆H₄Cl |
| (335) | H | CH₃ | —C(C₆H₅)(OH)— | C₆H₅ |
| (336) | H | CH₃ | —C(C₆H₅)(OH)— | 2-C₄H₃S |
| (337) | CH₃ | H | —CO— | p-C₆H₄F |
| (338) | CH₃ | H | —C(C₆H₅)(OH)— | p-C₆H₄F |
| (339) | CH₃ | H | —C(p-C₆H₄F)(OH)— | p-C₆H₄F |
| (340) | CH₃ | H | —CH(OH)— | p-C₆H₄F |
| (341) | H | F | —CO— | p-C₆H₄F |
| (342) | H | F | —C(C₆H₅)(OH)— | p-C₆H₄F |
| (343) | H | F | —C(p-C₆H₄F)(OH)— | p-C₆H₄F |
| (344) | H | Cl | —CH(OH)— | p-C₆H₄F |
| (345) | F | CH₃ | —CO— | p-C₆H₄F |
| (346) | F | CH₃ | —C(C₆H₅)(OH)— | p-C₆H₄F |
| (347) | F | CH₃ | —C(p-C₆H₄F)(OH)— | p-C₆H₄F |
| (348) | Cl | CH₃ | —CH(OH)— | p-C₆H₄F |

### EXAMPLES 349-416

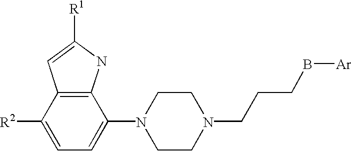

| | R¹ | R² | B | Ar |
|---|---|---|---|---|
| (349) | H | H | —CO— | p-C₆H₄F |
| (350) | H | H | —CO— | o-C₆H₄F |
| (351) | H | H | —CO— | m-C₆H₄F |
| (352) | H | H | —CO— | p-C₆H₄Cl |
| (353) | H | H | —CO— | m-C₆H₄Cl |
| (354) | H | H | —CO— | C₆H₅ |
| (355) | H | H | —CO— | 2-C₄H₃S |
| (356) | H | H | —CH(OH)— | p-C₆H₄F |
| (357) | H | H | —CH(OH)— | o-C₆H₄F |
| (358) | H | H | —CH(OH)— | m-C₆H₄F |
| (359) | H | H | —CH(OH)— | p-C₆H₄Cl |
| (360) | H | H | —CH(OH)— | m-C₆H₄Cl |
| (361) | H | H | —CH(OH)— | C₆H₅ |
| (362) | H | H | —CH(OH)— | 2-C₄H₃S |
| (363) | H | H | —C(p-C₆H₄F)(OH)— | p-C₆H₄F |
| (364) | H | H | —C(p-C₆H₄F)(OH)— | o-C₆H₄F |
| (365) | H | H | —C(p-C₆H₄F)(OH)— | m-C₆H₄F |
| (366) | H | H | —C(p-C₆H₄F)(OH)— | p-C₆H₄Cl |
| (367) | H | H | —C(p-C₆H₄F)(OH)— | m-C₆H₄Cl |
| (368) | H | H | —C(p-C₆H₄F)(OH)— | C₆H₅ |
| (369) | H | H | —C(p-C₆H₄F)(OH)— | 2-C₄H₃S |
| (370) | H | H | —C(C₆H₅)(OH)— | p-C₆H₄F |
| (371) | H | H | —C(C₆H₅)(OH)— | o-C₆H₄F |
| (372) | H | H | —C(C₆H₅)(OH)— | m-C₆H₄F |
| (373) | H | H | —C(C₆H₅)(OH)— | p-C₆H₄Cl |
| (374) | H | H | —C(C₆H₅)(OH)— | m-C₆H₄Cl |
| (375) | H | H | —C(C₆H₅)(OH)— | C₆H₅ |
| (376) | H | H | —C(C₆H₅)(OH)— | 2-C₄H₃S |
| (377) | H | CH₃ | —CO— | p-C₆H₄F |
| (378) | H | CH₃ | —CO— | o-C₆H₄F |
| (379) | H | CH₃ | —CO— | m-C₆H₄F |
| (380) | H | CH₃ | —CO— | p-C₆H₄Cl |
| (381) | H | CH₃ | —CO— | m-C₆H₄Cl |
| (382) | H | CH₃ | —CO— | C₆H₅ |
| (383) | H | CH₃ | —CO— | 2-C₄H₃S |
| (384) | H | CH₃ | —CH(OH)— | p-C₆H₄F |
| (385) | H | CH₃ | —CH(OH)— | o-C₆H₄F |
| (386) | H | CH₃ | —CH(OH)— | m-C₆H₄F |
| (387) | H | CH₃ | —CH(OH)— | p-C₆H₄Cl |
| (388) | H | CH₃ | —CH(OH)— | m-C₆H₄Cl |
| (389) | H | CH₃ | —CH(OH)— | C₆H₅ |
| (390) | H | CH₃ | —CH(OH)— | 2-C₄H₃S |
| (391) | H | CH₃ | —C(p-C₆H₄F)(OH)— | p-C₆H₄F |
| (392) | H | CH₃ | —C(p-C₆H₄F)(OH)— | o-C₆H₄F |
| (393) | H | CH₃ | —C(p-C₆H₄F)(OH)— | m-C₆H₄F |
| (394) | H | CH₃ | —C(p-C₆H₄F)(OH)— | p-C₆H₄Cl |
| (395) | H | CH₃ | —C(p-C₆H₄F)(OH)— | m-C₆H₄Cl |
| (396) | H | CH₃ | —C(p-C₆H₄F)(OH)— | C₆H₅ |
| (397) | H | CH₃ | —C(p-C₆H₄F)(OH)— | 2-C₄H₃S |
| (398) | H | CH₃ | —C(C₆H₅)(OH)— | p-C₆H₄F |
| (399) | H | CH₃ | —C(C₆H₅)(OH)— | o-C₆H₄F |
| (400) | H | CH₃ | —C(C₆H₅)(OH)— | m-C₆H₄F |
| (401) | H | CH₃ | —C(C₆H₅)(OH)— | p-C₆H₄Cl |
| (402) | H | CH₃ | —C(C₆H₅)(OH)— | m-C₆H₄Cl |
| (403) | H | CH₃ | —C(C₆H₅)(OH)— | C₆H₅ |
| (404) | H | CH₃ | —C(C₆H₅)(OH)— | 2-C₄H₃S |
| (405) | CH₃ | H | —CO— | p-C₆H₄F |
| (406) | CH₃ | H | —C(C₆H₅)(OH)— | p-C₆H₄F |
| (407) | CH₃ | H | —C(C₆H₅F)(OH)— | p-C₆H₄F |
| (408) | CH₃ | H | —CH(OH)— | p-C₆H₄F |
| (409) | H | F | —CO— | p-C₆H₄F |
| (410) | H | F | —C(C₆H₅)(OH)— | p-C₆H₄F |
| (411) | H | F | —C(p-C₆H₄F)(OH)— | p-C₆H₄F |
| (412) | H | Cl | —CH(OH)— | p-C₆H₄F |
| (413) | F | CH₃ | —CO— | p-C₆H₄F |
| (414) | F | CH₃ | —C(C₆H₅)(OH)— | p-C₆H₄F |
| (415) | F | CH₃ | —C(p-C₆H₄F)(OH)— | p-C₆H₄F |
| (416) | Cl | CH₃ | —CH(OH)— | p-C₆H₄F |

### EXAMPLE A

### Ampoules for Injection

A solution of 100 g of a compound of the general formula I and 5 g of disodium hydrogenphosphate is adjusted to pH 6.5 using 2 N hydrochloric acid in 3 l of double-distilled water, sterile filtered and filled into injection ampoules, and lyophilized. Sterile conditions were adhered to here. Each injection ampoule contains 5 mg of the active component of the, general formula I.

### EXAMPLE B

A mixture of 20 g of a compound of the general formula I is mixed with 100 g of soya lecithin and 1400 g of cocoa butter with warming and poured into hollows. Each suppository contains 20 mg of the active component.

### EXAMPLE C

A solution comprising 1 g of a compound of the general formula I, 9.38 g of NaH₂PO₄×2 H₂O, 28.48 g of Na₂HPO₄×12 H₂O and 0.1 g of benzalkonium chloride is prepared using 940 ml of double-distilled water. The solution is adjusted to pH 6.8 and made up to one litre with double-distilled water and sterilized by irradiation. This solution can be used in the form of eye drops.

### EXAMPLE D

### Ointment

500 mg of a compound of the general formula I are blended with 99.5 g of raw petroleum jelly under aseptic conditions.

### EXAMPLE E

### Tablets

100 g of a compound of the general formula I, 1 kg of lactose, 600 g of microcrystalline cellulose, 600 g of cornstarch, 100 g of polyvinyl-pyrrolidone, 80 g of talc and 10 g of magnesium stearate are mixed and pressed in a customary manner to give tablets such that one tablet contains 100 mg of the active component.

### EXAMPLE F

### Coated Tablets

Tablets are prepared as in Example 7 and then coated in a known manner with sucrose, maize starch, talc, tragacanth gum and colorants.

### EXAMPLE G

### Capsules

Hard gelatin capsules are filled with a compound of the general formula I in a known manner such that each capsule contains 5 mg of the active component.

### EXAMPLE H

### Inhalation Spray

14 g of a compound of the general formula I are dissolved in 10 l of isotonic saline solution. The solution is filled into commercially obtainable spray containers which have a pump mechanism. The solution can be sprayed into the mouth or into the nose. One puff of spray (approximately 0.1 ml) corresponds to a dose of 0.14 mg of a compound of the general formula I.

1. Arylpiperazine compounds of the formula I 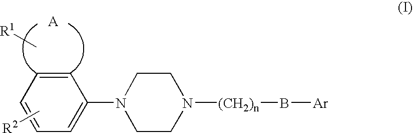
where
A is a fused heteroaromatic or heteroaliphatic ring comprising one or two nitrogen atoms,
B is —CO— or —CHOH— or —C(Ar)(OH)—
R¹ and R² independently of one another are H, alkyl, C₁-C₆ or halogen
Ar is phenyl or thiophene, which is unsubstituted or monosubstituted or polysubstituted by halogen, NO₂ or CN and
n is 1, 2, 3 or 4,
and their salts and solvates.

2. Compounds of the formula I according to one of the preceding claims, characterized in that the group 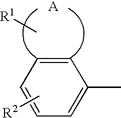
has one of the following meaning:

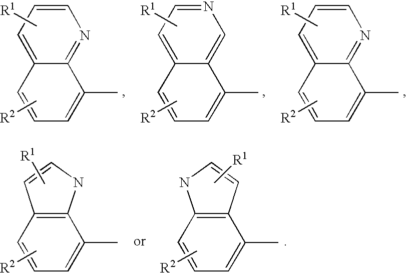

3. Compounds selected from the following group of compounds 1a to 1h: 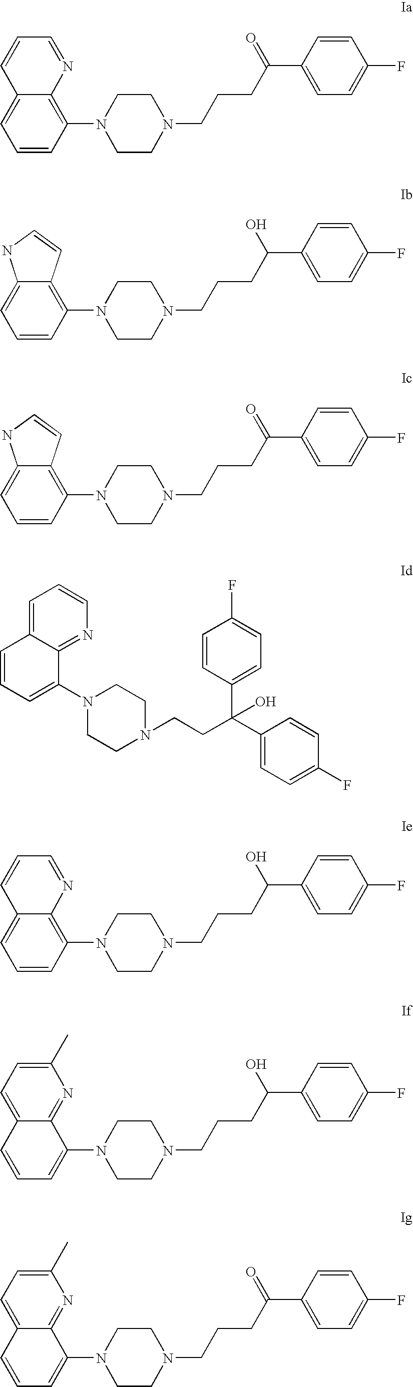 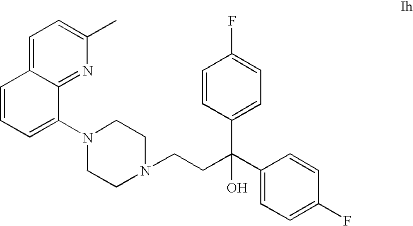
and their salts and solvates.

4. Compounds of the formula I according to claim 1 and their physiologically acceptable salts or solvates as pharmaceutical active compounds.

5. Compounds of the formula I according to claim 1 and their physiologically acceptable salts or solvates as D₂ receptor antagonists and/or 5HT_{1A} antagonists.

6. Compounds of the formula I according to claim 1 and their physiologically acceptable salts or solvates for use in the control of diseases.

7. Pharmaceutical preparation characterized in that it contains at least one compound of the formula I according to claim 1 and/or one of its physiologically acceptable salts or solvates.

8. Use of compounds of the formula I according to claim 1 and/or their physiologically acceptable salts or solvates for the production of a medicament.

9. Use of compounds of the formula I according to claim 1 and/or their physiologically acceptable salts or solvates for the production of a medicament for the treatment of illnesses of the central nervous system, in particular of mental disorders of the schizophrenia type and for the control of psychotic anxiety states.

10. Process for the preparation of compounds of the formula I and their salts and solvates, characterized in that a compound of the formula II 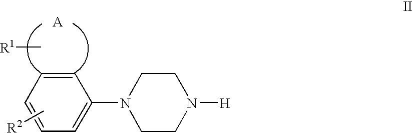
in which R¹, R² and A have the meaning indicated above, is reacted with a compound of the formula III 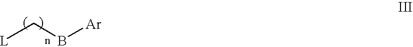
in which Ar, B and n have the meaning indicated above and L is a leaving group and, if B has the meaning —CO—, the group B is optionally hydrogenated, alkylated or arylated and, if appropriate, a basic or acidic compound of the formula I is converted into one of its salts or solvates by treating with an acid or base.
